(19) United States
(12) Patent Application Publication
(10) Pub. No.: US 2003/0170739 A1
(43) Pub. Date: Sep. 11, 2003

(54) Gene expression for analyzing photodamage

(75) Inventors: Susanne Teklits Iobst, Maywood, NJ (US); Kurt Matthew Schilling, Totowa, NJ (US); Charles Boyd, Honolulu, HI (US); Johann Urschitz, Honolulu, HI (US)

Correspondence Address: UNILEVER, PATENT DEPARTMENT, 45 RIVER ROAD, EDGEWATER, NJ, 07020, US

(73) Assignee: Unilever Home & Personal Care USA, Division of Conopco, Inc.

(21) Appl. No.: 10/265,509

(22) Filed: Oct. 7, 2002

Related U.S. Application Data
(60) Provisional application No. 60/337,856, filed on Nov. 8, 2001.

(51) Int. Cl. G01N 33/53; G01N 33/567

(52) U.S. Cl. 435/7.2

(57) ABSTRACT

The present invention relates to polynucleotide sequences in gene arrays that function as markers of photodamage and a personal care method of detecting photodamage using the markers.

This application claims priority under 35 U.S.C. § 119 from U.S. provisional application Serial No. 60/337,856, filed Nov. 8, 2001, and incorporated by reference herein.

### FIELD OF THE INVENTION

The present invention relates to polynucleotide sequences in gene arrays that function as markers of photodamage and a method of detecting photodamage using the markers.

### BACKGROUND OF THE INVENTION

All the genes of a cell comprise the genome. The human genome contains approximately 40,000 genes. However, in any given cell, only a fraction of these genes are expressed, or caused to manifest their effects in the phenotype. Phenotype is meant to refer to the visible properties of an organism that are produced by the interaction of the genotype and the environment. Therefore, in each cell type, only a fraction of human genomes are expressed at any one time. Each gene is expressed at a precise time and at a precise level.

Automated DNA sequencers have made it easier to determine the sequence of the genome of an organism. For example, the genomic sequences of Haemophilius influenzae, Mycoplasma genitalium, and Caenorhabditis elegans have been published, leading to the possiblity that the genomic sequence of higher organisms, such as humans, would be obtained (Fleischmann, R. D. et al. (1995) Science 269:496; Fraser, C. M. et al. (1995) Science 270:397; Hodgkin, J. et al. (1995) Science 270:410 ).

A typical mammalian cell of a given lineage expresses approximately 20,000-30,000 of the 40,000 odd germ line genes carried in its genome. Almost all cells universally express many of the genes, which are called “housekeeping” genes. Examples of housekeeping genes include genes encoding enzymes involved in glycolysis or proteins involved in cell structure. However, it is the non-universally expressed genes that differentiate cells from each other. As cells mature into differentiated cells, certain non-constitutively expressed genes are turned on and off at different stages. Thus, the differences in gene expression patterns between cells make, for example, a nerve cell different from a blood cell.

Under abnormal cellular conditions such as those in individuals with disease or disorders, the pattern of gene expression within individual cells may be changed compared to the expression pattern seen under normal non-disease conditions. A change in gene expression may be an effect or the cause of a disease or abnormality, such as in, for example, a tumor cell. Whereas some diseases may be understood as caused by mutations in particular genes and thus potentially be detected by examining the genomic sequence, many diseases and disorders involve a malfunction in the level of expression of genes which cannot be detected by sequencing the genome but can only be detected by identifying the gene expression patterns of the cells. Therefore, in order to understand the function of specific cell types in an organism (at a given period of their lifetime) or to understand the progression of a disease or disorder, it is necessary to understand the expression status of individual genes within these specific cell types at different stages of the organism's development.

Aging of the skin is thought to consist of two processes taking place simultaneously. The first process is intrinsic, chronologic aging and similar perhaps to aging of other tissues (Uitto, 1986). The second process is photoaging, an environmentally-induced remodeling of the dermis that arises as a result of repeated exposure of skin to sunlight. Although recent studies (Varani et al., 1998; Varani et al., 2000) have shown that both intrinsic aging and photoaging share some common characteristics such as decreased procollagen gene expression and increased expression of genes encoding several matrix metalloproteinases, it has been suggested that photoaging is the predominant contributing factor to the prematurely aged appearance of sun-exposed skin (Yaar and Gilchrest, 1998).

Clinically, sun-damaged skin is characterized by wrinkling, loss of resilience and an altered texture (Kligman, 1989; Taylor et al., 1990). Early studies attribute these features primarily to changes in the dermis, as histopathologic analyses have revealed alterations in a variety of extracellular matrix proteins within the dermis of sun-exposed skin. The most prominent of these dermal changes is the marked accumulation of elastic fibers with a clearly altered morphology in the superficial dermis of sun-exposed skin. This accumulation of aberrant dermal elastic fibers following sun-exposure has been referred to as solar elastosis (Gilchrest, 1989).

The cellular mechanisms leading to solar elastosis are not understood and indeed, controversial findings concerning the synthesis of elastic fibers during solar elastosis have been reported. Several reports have demonstrated that elastic fibers deposited during solar elastosis consist of the same components as normal elastic fibers and these include elastin (the insoluble and crosslinked protein that makes up the amorphous component of elastic fibers) and fibrillin, the major microfibrillar component of elastic fibers. In response to UVA and/or UVB radiation, keratinocytes secrete many mediators that could stimulate fibroblast synthetic activity and some of them, eg. TGF-β, IL-1β and IL-10, have been shown to increase the promoter activity of the elastin gene, steady state mRNA levels and increased elastin accumulation (Kahari et al., 1992; Mauviel et al., 1993; Reitamo et al., 1994). While Bernstein et al. (1994) have noted increased elastin mRNA levels in sun-damaged skin, Werth and co-workers however have (Werth et al., 1997) reported no difference in steady-state levels of elastin mRNA during solar elastosis. The latter finding is in agreement with an earlier study which indicated that a post-transcriptional mechanism leads to an increased translational efficiency responsible for elastin accumulation in response to ultraviolet-irradiation in the absence of increased mRNA levels (Schwartz et al., 1995). These results implicate that aberrant expression of genes encoding structural proteins of elastic fibers, as a consequence of UV-exposure, could be the basis of solar elastosis. Indeed, several reports have demonstrated changes in steady-state mRNA levels not only of elastin but also fibrillin (Bernstein et al., 1994). Additional observations have also noted changes in the levels of elastic fiber proteins such as lysyl oxidase, the copper-dependent amine oxidase responsible for the catalysis of elastin crosslinking (Smith-Mungo and Kagan,1998).

Other changes in extracellular matrix proteins in response to UV-irradiation have also been demonstrated. For example the amount of collagen fibrils have been shown to be drastically decreased in photoaged skin. This change is not accompanied by a change in collagen mRNA levels, suggesting that degradation of collagen fibrils is associated with UV exposure (Bernstein et al., 1996). To explain these changes in collagen deposition, Voorhees and coworkers have proposed that UV irradiation triggers an increase of growth factor and cytokine receptor synthesis in fibroblasts and keratinocytes. This increased receptor synthesis in turn, leads to an activation of the transcription factor AP-1 (Fisher et al., 1996; Fisher and Voorhees, 1998) through a MAP kinase (mitogen-activated protein kinase) signaling cascade and an increase in the expression of genes encoding several collagen-degrading matrix metalloproteinases (Fisher et al., 1996) and a decreased expression of the genes encoding type I and III procollagen.

While an attractive hypothesis, this model for an AP-1 activation of matrix metalloproteinase gene expression does not accommodate for the many other changes in extracellular matrix that have been shown to be associated with UV exposure. Moreover it is very likely that the pathobiology of sun-damaged skin arises through a complex interaction of multiple direct and indirect changes in gene expression in the dermis and epidermis, AP-1 activation representing just one of these changes.

This complex cascade of events associated with sun damage is not well understood. To identify changes in transcript profiles in response to sun exposure, researchers have used many techniques such as isolating proteins from various cells and comparing the abundance of each of the proteins. Another method involves the use of antibodies to probe populations of peptides produced from mRNA pools. Therefore, “libraries” of synthetic polypeptides corresponding to the polypeptides coded for by mRNA molecules are produced and then probed by individual antibodies, as described in U.S. Pat. No. 5,242,798.

In parallel to progress made in determining which genes are expressed by a given tissue or cell, major advances are being made in the biotechnology industry in the design and production of gene “array” technology. Techniques such as SAGE (Serial Analysis of Gene Expression) can be used to generate data on keratinocytes (or epidermis) and thereby develop the gene arrays.

Gene arrays are solid phase systems harboring immobilized nucleotide sequences that represent up to thousands of individual genes of interest (of known or unknown function). Such arrays can be utilized to test extracts of tissue or cell cultures to determine which genes are turned on or off in response to treatments, insults, age, gender, ethnicity, drugs, foods, and cosmetics. However, the methods available in the prior art still make it difficult to track the expression of even small numbers of genes in laboratory models or in human tissue.

Several patents pertain to the use of the SAGE technique, or to the making of arrays, as well as patents protecting instruments designed to make and process arrays. For example, EP 799897 discloses methods and compositions for selecting tag nucleic acids in probe arrays. WO 9743450 discloses hybridization assays on oligonucleotide arrays. WO 9815651 discloses methods for identifying antisense oligonucleotide binding. However, none of the known patents disclose the identification and use specific gene arrays for identification of photodamage.

As used herein, the term “comprising” means including, made up of, composed of, consisting of and/or consisting essentially of. Except in the operating and comparative examples, or where otherwise explicitly indicated, all numbers in this description indicating amounts or ratios of material or conditions of reaction, physical properties of materials and/or use are to be understood as modified by the word “about.”

The term “skin” as used herein includes the skin on the face, neck, chest, back, arms, hands, legs, and scalp. The terms “epidermis” or “keratinocytes” are viewed as being encompassed by the term “skin.”

### SUMMARY OF THE INVENTION

A personal care method of detecting photodamage comprising the steps of:

(a) using at least one marker of photodamage, the marker selected from one or more sequences selected from the group consisting of sequence No. 51, sequence No. 52, sequence No. 53, sequence No. 54, sequence No. 55, sequence No. 56, sequence No. 57, sequence No. 58, sequence No. 59, sequence No. 60, sequence No. 61, sequence No. 62, sequence No. 63, sequence No. 64, sequence No. 65, sequence No. 66, sequence No. 67, sequence No. 68, and sequence No. 69; and

(b) detecting a change in the marker to determine the presence of photodamage.

### DETAILED DESCRIPTION OF THE INVENTION

The present invention relates to polynucleotide sequences in gene arrays that function as markers of photodamage and a method of detecting photodamage using the markers.

As used herein, the following terms are to be understood as follows.

“Medical Applications” are Devices and compositions which are distributed solely by prescription or solely to the medical profession.

“Personal Care Applications” are Devices and compositions for the cleaning and care of human skin, except Medical Applications.

A “gene” is a unit of inheritable genetic material found in a human chromosome.

The recurring structural units of all nucleic acids are eight different nucleotides; four kinds of nucleotides are the building blocks of DNA, and four others are the structural units of RNA. For example, the four-letter language of DNA is translated into the twenty-letter language of protein.

“Oligonucleotides” are oligomer fragments comprised of two or more deoxyribonucleotides or ribonucleotides, preferably more than three.

A “polynucleotide sequence” is a polymeric chain of mononucleotides in a given order. Polynucleotide sequence is reported from 5′-3′ end or the complementary strand from 3′-5′.

An “Expressed Sequence Tag” (“EST”) is a nucleotide sequence which includes a sufficient number of base pairs such that it uniquely defines a cDNA (complementary deoxyribonucleic acid) sequence. The EST is both isolated and purified.

“Isolated” refers to nucleic acid separated from other cellular components.

“Purified” refers to an isolated nucleic acid mixture from which as much other material has been removed, so as to leave only nucleic acid.

Gene expression analysis is a tool that can be utilized to identify those markers that are indicative of specific skin conditions such as photodamage or dry skin. Photodamage, dry skin, oily skin, and other “cosmetic” skin conditions are not well understood biologically. Traditionally these conditions are studied by addressing one biological pathway at a time. The present invention provides for the application of SAGE techniques, described in more detail below, to comprehensively study skin conditions to elucidate new pathways. The present invention provides polynucleotide sequences which are indicative of a particular skin condition. Specifically, the present invention provides specific ESTs (sets of genes) that are modulated in photodamage and therefore can be used as markers of photodamage. The ESTs or markers of the present invention have never before been known to be important or used for identifying photodamage.

A preferred method of identifying the polynucleotide sequence is through the use of SAGE (Serial Analysis of Gene Expression), as described in U.S. Pat. No. 5,695,937. This technique allows the analysis of a large number of transcripts. Essentially, cDNA oligonucleotides are produced. A first defined nucleotide sequence tag is then isolated form a first cDNA oligonucleotide and a second defined nucleotide sequence tag is isolated from a second cDNA oligonucleotide. The nucleotide sequence of the first and second tags are determined so that the tags correspond to an expressed gene.

The present invention provides a method of using EST's, as well as the proteins they code, for identifying photodamage. The method comprises a first step of selecting a first epidermal sample having at least one sequence and selecting a second epidermal sample having the same sequence. The first epidermal sample is compared to the second epidermal sample to determine whether there is a change in the sequence. If there is a change in the sequence, then photodamage exists in the second epidermal sample. The same method is applicable to samples of the dermis or total skin.

A Comparison of Post-and Pre-Auricular Skin SAGE Libraries

In order to identify genes that were differentially expressed in sun-exposed skin, the SAGE libraries for pre-and post-auricular skin were compared. A small but significant fraction of the analyzed SAGE sequence tags showed marked differences in copy number between pre-and post-auricular skin. 19 unique tags were found at significantly lower levels (at least 4-fold lower) in sun-exposed pre-auricular skin, whereas 15 showed at least 4-fold higher levels in pre-auricular skin. Tables 4 and 5 list these tags with notably different copy numbers. Of these tags, 24 could be uniquely matched to the UniGene database and ten tags had either multiple matches or no matches. Three of these unmatched tags have sequences that consist primarily of multiple deoxyadenosine residues: Tag Seq. No. 51 (CAAAAAAAAA) and Tag Seq. No. 65 (GGAAAAAAAAA) in Table I; Tag Seq. No. 77 (TAAAAAAAAAA) in Table II. Another four tags reliably matched two different genes, 1 tag showed no significant similarity with any oriented GenBank cDNA sequences but had been found in other SAGE libraries and 1 tag had no match to UniGene or to any other SAGE library. The remaining unidentified tag represented an Alu repeat sequence and resulted therefore in numerous matches.

Of the 24 uniquely matched tags, we observed a 7-fold higher Keratin 1 tag number in pre-auricular skin. We also found elevated copy numbers for tags derived from several other genes in sun-damaged skin (Table II) and these include:

1. The psoriasin gene encodes a member of the S100 calcium binding protein family and tags derived from this gene were found in a 4-fold higher level in pre-auricular skin. Psoriasin protein and mRNA levels have been reported to be raised in UVB exposed skin in vivo up to 10 days post-exposure (Di Nuzzo et al., 2000). Furthermore, psoriasin has previously been shown to be present in all layers of psoriatic epidermis, has been shown to be associated with epidermal fatty acid binding protein (EFABP) and both the genes encoding psoriasin and EFABP are known to be up-regulated in psoriasis (Hagens et al., 1999). EFABP gene expression has also been shown to be induced in human skin by topical application of retinoic acid (Larsen et al., 1994). The SAGE data of the present invention revealed a 6-fold higher tag count for EFABP mRNA in sun-exposed skin as compared to normal skin.

2. The mRNA encoding Insulin-like growth factor binding protein 6 (IGFBP-6), is represented by 6 tags in pre-auricular skin and by 1 tag in post-auricular skin; IGFBP-6 binds Insulin-like growth factor II (IGF-II) with high affinity and this binding inhibits IGF-II action. Three groups of IGFBP proteases (matrix metalloproteinases, kallikreins and cathepsins) cleave the IGFBP-IGF complex and have been shown to release a functional IGF from its binding protein. IGFBP-6 has been associated with quiescent, non-proliferating cells, suggesting that IGFBP-6 acts as an autocrine growth inhibitor (Kato et a., 1995). Kelley et al. (Kelley et a., 1996) have suggested that IGFBPs may also have additional intrinsic biological activities, independently of IGFs.

3. The mRNA for calmodulin-like skin protein (CLSP), is represented at a 16 to 4 tag ratio between sun-damaged and sun-protected skin. CLSP is a recently identified protein that was reported to be particularly abundant in the epidermis. CLSP gene expression moreover has been shown to be directly associated with keratinocyte differentiation (Mehul et al, 2000).

4. Macrophage migration inhibitory factor (MIF) mRNA was 4-fold up-regulated in pre-auricular skin. MIF, originally reported be released by activated T-cells, inhibits the migration of macrophages and activates macrophages at inflammatory loci. In addition, a previous study implicated MIF as a regulator in epidermal immunity and cell differentiation (Shimizu et al., 1996). UVB irradiation has been shown to induce MIF production in human epidermal keratinocytes in vivo and in vitro (Shimizu et al., 1999). In addition MIF is also thought to be involved in psoriasis as MIF levels are elevated in psoriatic plaques (Steinhoff et al., 1999). MIF appears therefore to function as an inhibitor of anti-inflammatory action by coordinating several pro-inflammatory cytokines, as well as regulation of the immunosuppressive effects of steroids on immune cell activation and cytokine production.

5. 4 SAGE tags for the Testis enhanced gene transcript (TEGT) were identified in the pre-auricular skin library of the present invention, whereas only 1 tag was detected among all the post-auricular tags. TEGT was found to be identical to bax-inhibitor 1 (BI-1), a recently described repressor of the pro-apoptotic protein bax (Xu and Reed, 1998). Although the mechanism of apoptosis inhibition is not yet defined, it has been shown that BI-1 has no significant impact on the levels of bax. Therefore it was suggested that BI-1 inhibits bax indirectly, possibly by substituting for the anti-apoptotic protein bcl-2.

6. The number of tags representing cellugyrin mRNA increased 4-fold in pre-auricular skin. Cellugyrin is a ubiquitously expressed member of the synaptic vesicle protein family of synaptogyrins, which are essential for the regulation of synaptic vesicle trafficking (Janz and Sudhof, 1998). In adipocytes, for example, insulin activates the translocation of glucose transporter 4 (Glut4)-containing membrane vesicles from intracellular compartments to the plasma membrane, which ultimately leads to an increased glucose uptake. As insulin stimulation does not initiate a re-distribution of cellugyrin-positive Glut4 vesicles to the plasma membrane, it is believed that these vesicles do have unique functional properties, independent from those glut4 vesicles that translocate to the plasma membrane (Kupriyanova and Kandror, 2000).

7. The mRNA for imogen 38 (mitochondrial 38 kD islet antigen) is also represented by a 4-fold increased tag number in pre-auricular skin, This antigen is one of the molecular targets of autoreactive T cells in type I diabetes (insulin-dependent diabetes mellitus).

Reduced tag numbers from mRNAs encoding known proteins in sun-exposed skin (Table I) include:

1. Cathepsin D (Sequence No. 55) showed the most significant decrease (5-fold less) in mRNA steady state levels in pre-auricular skin. Cathepsin D is a lysosomal aspartic proteinase known to be present in the epidermis as well as many other tissues. The chronology of activation and degradation of this protein has been shown to be connected to stages of cellular differentiation and the expression of Cathepsin D in the epidermis resembles that of other structural proteins such as keratin 10, involucrin and transglutaminase, in response to calcium concentration changes (Horikoshi et al., 1998).

2. Ladinin mRNA (Sequence No. 56 in Table I below), which showed a five-fold decrease of representative SAGE tags in our pre-auricular library in comparison to the post-auricular library, is an anchoring-filament associated protein and is one of several basement-associated proteins that contribute to autoimmune disorders such as linear IgA disease (Moll and Moll, 1998).

3. Sequence No. 57 in Table I below was found 9 times in post-auricular skin and only 2 times in pre-auricular skin. This tag matched two different UniGene database entries, one for an mRNA encoding lecithin-cholesterol acyltransferase (LCAT) and a second entry for an mRNA encoding Bcl-2-antagonist (Bak). LCAT converts cholesterol to cholesteryl ester and is the key enzyme in maintaining cholesterol homeostasis in blood. Infection or inflammation perturb lipoprotein metabolism and plasma concentrations of lipids and lipoproteins as well as LCAT are known to change under these conditions (Khovidhunkit et al., 2000). Bak, on the other hand, is a proapoptotic protein that shares a high sequence homology with bax. Both proteins are thought to oligomerize in mitochondrial membranes, forming pores that facilitate cytochrome c efflux (Korsmeyer et al., 2000) and trigger an apoptosis cascade.

4. A 4-fold lower mRNA level for the mRNA encoding zyxin was detected in sun-damaged skin as compared to normal skin. Zyxin is a focal adhesion phosphoprotein reported to be expressed in all layers of the epidermis (Leccia et al., 1999). Moreover zyxin is also found in fibroblasts where the protein has been shown to be colocalized both with cell-substratum and also with cell-cell adherens junctions. Zyxin shares architectural characteristics (such as LIM domains, a double zinc-finger motif) with signal transducers involved in developmental regulation and previous work has suggested that zyxin may also be involved in the regulation of cell proliferation and differentiation (Beckerle, 1997).

5. An mRNA encoding the calcium ion binding protein S100 A3 showed decreased levels in the SAGE library derived from sun-damaged skin. As with psoriasin, S100 A3 is a member of the S100 Calcium binding gene family. Significant expression of the gene encoding S100A3 in mouse is limited to the hair follicle and the timing of expression of this gene is synchronized with the neonatal and adolescent phases of the hair growth cycle (Kizawa et al., 1998).

6. A reduced number of tags in sun-exposed skin was observed for another Ca²⁺ binding protein called cartilage oligomeric matrix protein (COMP). COMP is an extracellular matrix glycoprotein expressed not only in cartilage and ligaments but also in human dermal fibroblasts in vitro (Dodge et al., 1998) and cultured human vascular smooth muscle cells (Riessen et al., 2001). Mutations in COMP have been shown to result in decreased calcium binding ability which ultimately leads to the skeletal disorder pseudoanchodroplasia (PSACH) (Maddox et al., 2000). However, in other respects very little is known about the function of COMP.

A reduction in the number of tags derived from ribosomal RNAs (ACATCATCGAT-Seq. No. 53 and ACTCCAAAAAA-Seq. No. 54) and from mRNAs encoding unknown proteins (CAAAAAAAAAA-Seq. No. 51 and ACGTTAAAGA-Seq. No. 52) in sun-exposed pre-auricular skin was observed.

TABLE I

Genes down-regulated in pre-auricular skin

| | | | | | |
|---|---|---|---|---|---|
| Seq. No. | Tag Sequence^{a} | Post | Pre | Post/Pred | UniGene match^{b} (Accession No.)^{c} |

| | | | | | |
|---|---|---|---|---|---|
| 51 | CAAAAAAAAAA | 7 | 1 | 7.0 | Multiple matches |
| 52 | ACGTTAAAGA | 6 | 1 | 6.0 | Tag not found in oriented Gen Bank cDNA |
| | | | | | sequences |
| 53 | ACATCATCGAT | 5 | 1 | 5.0 | Ribosomal protein L12 (L06505) |
| 54 | ACTCCAAAAAA | 5 | 1 | 5.0 | Ribosomal protein S15 (AA079663)/ |
| | | | | | IMAGE clone 3840457 (BC012990) |
| 55 | GAAATACAGTT | 5 | 1 | 5.0 | Cathepsin D (M11233) |
| 56 | GCCAGGAGCTA | 5 | 1 | 5.0 | Ladinin 1/ESTs, Highly similar to ATIC |
| | | | | | (U42408/A1214479) |
| 57 | CTCCTCACCTG | 9 | 2 | 4.5 | BCL2-antagonist (U16811) ribosomal |
| | | | | | protein L13A (NM012423) |
| 58 | CAATAAACTGA | 4 | 1 | 4.0 | Putataive translation initiation factor |
| | | | | | (AA009621) |
| 59 | CAGCTCACTGA | 4 | 1 | 4.0 | Ribosomal protein L14 (D87735) |
| 60 | CAGGACCTGGT | 4 | 1 | 4.0 | Tag not found in oriented GenBank cDNA |
| | | | | | sequences |
| 61 | CCCAACGCGCT | 4 | 1 | 4.0 | Hemoglobin alpha 1 and alpha 2 (J00153) |
| 62 | CCCTGGCAATG | 4 | 1 | 4.0 | Uncharacterized hematopoietic |
| | | | | | stem/progenitor cells protein MDS027 |
| | | | | | (Af161418) |
| 63 | CTGCCAAGTTG | 4 | 1 | 4.0 | Zyxin (U15158) |
| 64 | GCAAAACCCCG | 4 | 1 | 4.0 | Multiple matches |
| 65 | GGAAAAAAAAA | 4 | 1 | 4.0 | Multiple matches |
| 66 | GGGGCAGGGCC | 4 | 1 | 4.0 | Eukaryotic translation initiation factor 5A |
| | | | | | (AW505485) |
| 67 | GTGCACTGAGC | 4 | 1 | 4.0 | Major histocompatibility complex, class I A |
| | | | | | and I C (M11887; M11886) |
| 68 | TCTCCCACACC | 4 | 1 | 4.0 | Calcium-binding protein S100 A3 (N002960) |
| 69 | CGGGGTGGCCG | 4 | 0 | 4.0 | Cartilage oligomeric matrix protein (L32137) |

^{a}Tags have been ranked by fold down-regulation, as indicated by a Post/Pre ratio.
^{b}The accession number or numbers indicate a representative EST derived from the corresponding UniGene cluster.
No accession number has been provided for tags, with either multiple matches or an EST match.
^{c}In order to avoid division by zero, we used a tag value of one for tags that were not detected at all.

TABLE II

Genes up-regulated in pre-auricular skin

| | | | | | |
|---|---|---|---|---|---|
| Seq.# | Tag Sequence^{a} | Pre | Post | Pre/Postd | UniGene match^{b} (Accession No.)^{c} |

| | | | | | |
|---|---|---|---|---|---|
| 70 | ACATTTCAAAG | 7 | 1 | 7.0 | keratin 1 (AA024512) |
| 71 | CAGCTATTTCA | 6 | 1 | 6.0 | fatty acid binding protein 5 (AF181449) |
| 72 | GGCCCCTCACC | 6 | 1 | 6.0 | insulin-like growth factor binding protein 6 |
| | | | | | (M69054) |
| 73 | ATCCGCGAGGC | 16 | 4 | 4.0 | calmodulin-like skin protein (AF172852) |
| 74 | AACGCGGCCAA | 8 | 2 | 4.0 | macrophage migration inhibitory factor |
| | | | | | (L10612) |
| 75 | GAGCAGCGCCC | 8 | 2 | 4.0 | S100 calcium-binding protein A7 |
| | | | | | (psoriasin 1) (M86757) |
| 76 | AAGAAGATAGA | 4 | 1 | 4.0 | ribosomal protein L23a/(U43701) |
| 77 | TAAAAAAAAAA | 4 | 1 | 4.0 | multiple matches |
| 78 | TCAGACTTTTG | 4 | 1 | 4.0 | diacylglycerol O-acyltransferase (NM |
| | | | | | 032564) |
| 79 | TTGGTGAAGGA | 4 | 1 | 4.0 | beta 4 thymosin (M17733) |
| 80 | AACTAACAAAA | 4 | 0 | 4.0 | ribosomal protein S27a (X63237) |
| 81 | CAATAAATGTT | 4 | 0 | 4.0 | ribosomal protein L37 (D23661) |
| 82 | GCTCCCAGACT | 4 | 0 | 4.0 | synaptogyrin 2 (AJ002308) |
| 83 | GGAAGTTTCGA | 4 | 0 | 4.0 | mitochondrial ribosomal protein |
| | | | | | 64(AB049959) |
| 84 | TCAAAAATATA | 4 | 0 | 4.0 | mitochondrial ribosomal protein S31 |
| | | | | | (NM005830) |

^{a}Tags have been ranked by fold up-regulation, as indicated by a Pre/Post ratio.
^{b, c,}are as described in the legend to Table I.

### EXAMPLE 1

To study the phenotypic changes in human skin associated with repeated sun exposure at the transcriptome level, we have undertaken a comparative Serial Analysis of Gene Expression (SAGE) of sun-damaged pre-auricular skin and sun-protected post-auricular skin as well as sun-protected epidermis. SAGE libraries, containing multiple mRNA-derived tag recombinants, were made to polyA(+) RNA isolated from human post-auricular skin and pre-auricular skin, as well as epidermal nick biopsy samples. 5,330 mRNA-derived cDNA tags from the post-auricular SAGE library were sequenced and these tag sequences were compared to cDNA sequences identified from 5,105 tags analyzed from a pre-auricular SAGE library. Of the total of 4,742 different tags represented in both libraries we found 35 tags with at least a 4-fold difference of tag abundance between the libraries. Among the mRNAs with altered steady-state levels in sun-damaged skin, we detected those encoding keratin 1, macrophage inhibitory factor and calmodulin-like skin protein. In addition, a comparison of cDNA sequences identified in the SAGE libraries obtained from the epidermal biopsy samples (5,257 cDNA tags) and from both full-thickness skin samples indicated that many genes with altered steady-state transcript levels upon sun-exposure were expressed in epidermal keratinocytes. These results suggest a major role for the epidermis in the pathomechanism of largely dermal changes in chronically sun-exposed skin.

Establishment of Gene Array

Protocol for Sequencing Tags:

The sequence analysis of the obtained ditag concatamers was performed using an ABI Prism 310 Genetic Analyzer, Perkin Elmer, Applied Biosystems, Shelton, Conn. This system is an automated instrument capable of determining base sequences or size and quantity of DNA fragments. It employs a combination of polyacrylamide capillary electrophoresis with multi-color fluorescent DNA detection.

The used BigDye Terminator Cycle Sequencing Ready Reaction Kit (PE Cat.#403044) Analyzer, Perkin Elmer, Applied Biosystems, Shelton, Conn. relies on the so called thermal cycle sequencing, a method that combines Sanger's fluorescent dideoxy sequencing procedure with a linear amplification of the DNA template.

Per sequencing reaction typically 1 μl (˜100 ng) of the concatamer-insert-size-check PCR reaction were added to 200 μl PCR tube containing 4 μl of Ready reaction Mix (Amplitaq FS, sequencing buffer and fluorescently labeled ddNTPs), 3.2 pmol M13 Reverse primer and 11 μl of ddH₂O. This solution was mixed and the cycle sequencing reaction was performed on an ABI GeneAmp PCR System 9700, Perkin Elmer, Applied Biosystems, Shelton, Conn., under the following conditions: 25 cycles with 96° C. for 10 seconds, 55° C. for 5 seconds and 60° C. again for 4 minutes.

The obtained extension products were purified by adding 2 μl of 3M sodium acetate and 50 μl of 95% ethanol to the tube. Following a precipitation of at least 15 minutes this mixture was spun for 20 minutes at maximum speed, the supernatant carefully aspirated and the remaining pellet washed twice with 70% ethanol. The pellet was dried and redissolved in 20 μl of Template Suppression reagent (PE Cat.#401674). Batches of up to 96 of these tubes were loaded onto the ABI Prism 310 Genetic Analyzer.

Conversion of Tags to Genes:

10-11 base pairs that constitute the SAGE tag were compared against the NCBI SAGE database (http://www3.ncbi.nim.nih.gov/SAGE/). The option of tag to gene mapping was selected. This identified the UniGene Cluster that the tag matched. It also identified the sequence or sequences that matched this ID. The individual sequences containing that tag were then extracted from the NCBI and a Multiple Sequence Alignment was completed. The longest clone was then reverse transcribed to yield the putative primary structure of the protein.

Northern Blot Analysis:

To confirm the expression changes in photodamage noted by SAGE, a confirmatory Northern blot was done using 3 different messages. These are shown in Table A below. They correspond well to the relationship determined by SAGE. Keratin I increased by a factor of 7, MIF migratory inhibitory factor increased by a factor of 4, and hrp S9 (human ribosomal protein S9) increased by a factor of 2.2.

Radiolabeled cDNA complementary to mRNAs for keratin 1, macrophage migration inhibitory factor (MIF) and human ribosomal protein S9 (hrpS9) were used to determine the levels of these mRNAs in poly(A⁺)RNA from the same pre- and post-auricular skin samples used to construct the SAGE libraries. Relative to the levels of a control GAPDH mRNA, the levels of keratin 1 mRNA, MIF mRNA and hrpS9 in pre- and post-auricular skin (Table A below) were consistent with our SAGE tag recovery data.

TABLE A

| | Fold increase | |
|---|---|---|
| | in photodamaged skin | |
| Gene | SAGE | Northern Blot |
| Keratin 1 | 7 | 4 |
| MIF | 4 | 2 |
| hrp S9 | 2.2 | 1.6 |
| G3PDH | 1 | 1 |
| CLSP | 4 | 2 |

### EXAMPLE 2

The following example provides data on sequencing of tags.

SAGE Analysis.

SAGE analysis was performed as described in Velculescu et al 1995. Essentially, double-stranded CDNA was synthesized from mRNA using a biotinylated oligo dT primer and then digested with Nla III. The biotinylated 3′ most cDNA fragments were isolated with magnetic streptavidin beads (Dynal, Oslo, Norway) and divided into two separate aliquots. Two different oligonucleotide linkers, containing a Bsmf I recognition site, a Nla III recognition site and PCR priming site, were ligated to DNA in each sample. Following Bsmf I digestion, the tags were ligated, the ditag products were PCR amplified, isolated by Nla III digestion, concatamerized and consecutively cloned into a pZero vector (Invitrogen, Carlsbad Calif.). Individual bacterial colonies containing recombinant plasmids were checked for insert sizes by PCR using M13 forward and M13 reverse primers. Insert-derived PCR products of at least 400 bp were then sequenced with the BigDye Terminator Kit (Perkin Elmer, Foster City, Calif.) and a 310 ABI automated DNA sequencer. Sequences were analyzed by the SAGE 2000 software program (version 4.12) which compares tags to the Genbank/EMBL databases and identifies and excludes duplicate ditags and tags derived from linkers. Tags originating from differentially expressed mRNAs were additionally analyzed with NCBI's SAGEmap “tag to gene” software.

SAGE Libraries

Three different SAGE libraries were constructed using poly(A⁺) RNA isolated from pre-auricular skin and post-auricular skin from a single donor and pooled epidermal nick biopsies. Upon subtracting tags derived from linkers, we generated 4,830 SAGE tags derived from pre-auricular skin and 4,990 tags derived from post-auricular skin. In addition, 5,215 SAGE tags derived from human epidermis were generated. Collectively, these 15,035 tags represented 6,598 unique genes.

Analysis of SAGE Tags from Post-Auricular Skin

Of the 2,858 unique tags obtained from post-auricular skin, 127 tags were observed at least five times and the total number of these repetitive tags represented 32% of the total tag number. 2,254 tags of the remaining low abundance tags were detected only once. Table Ill lists the 50 most abundant post-auricular SAGE tags that were detected, together with the frequency of these tags, reliable UniGene matches and a corresponding GenBank accession number. Tags originating from mitochondrial DNA were excluded. Whenever available, the 15^{th} base in the SAGE tag sequence (CATG+11 bp) was used to discriminate between multiple matches for the same tag. All tags except for two (tag 13 and tag 33) could be assigned to at least one gene. Tag 13 (ACTTTTTCAA) had no reliable matches to any UniGene cluster whereas tag 33 (ACCTCCACTG) could only be assigned to a cluster of ESTs in the UniGene database. Furthermore, according to the NCBI SAGE database, tag 33 has only been found, at low copy number, in one other SAGE library which had been generated from a primary ovarian tumor. 12 tags had multiple assignments; 5 of these tags matched sequences derived from two different genes and 7 originated from more than 2 different genes. Many of these abundant tags were derived from genes that are known to be widely expressed in various cell types, especially genes encoding ribosomal proteins, genes involved in protein synthesis (elongation factor 1), cytoskeletal genes (lamin A/C) and genes active in energy metabolism (glucose phosphate isomerase). Tags matching mRNAs derived from genes known to be specifically expressed in skin were also found. Among the most highly abundant of these skin-specific SAGE tags, we detected tags derived from mRNAs encoding several keratins as well as galectin 7 and calgranulin, all of which are typically found in full thickness skin.

TABLE III

The 50 most abundant tags from a human
post-auricular skin SAGE library

| | | | |
|---|---|---|---|
| | Tag | | |
| Tag Sequence | Count¹ | Accession No.^{b} | UniGene match^{c} |

| | | | | |
|---|---|---|---|---|
| 1 | CCCGTCCGGA | 63 | Al291979 | ribosomal protein L13 |
| 2 | TGCACGTTTT | 45 | X03342 | ribosomal protein L32 |
| 3 | CGCCGCCGGC | 38 | U12465 | ribosomal protein L35 |
| 4 | CGCTGGTTCC | 34 | L05092 | ribosomal protein L11 |
| 5 | GAGGGAGTTT | 31 | U14968 | ribosomal protein L27a |
| 6 | GGACCACTGA | 31 | M90054 | ribosomal protein L3 |
| 7 | AGGCTACGGA | 30 | AA045770 | ribosomal protein L13a |
| 8 | GCCCCTGCTG | 30 | M21389 | keratin 5 |
| 9 | GTGAAACCCC | 29 | | multiple matches |
| 10 | GGCAAGCCCC | 27 | AF107044/AL0227 | SRY-box 21/ribosomal protein L10a |
| | | | 21 | |
| 11 | ACGCAGGGAG | 23 | AF187554/ | glucose phosphate isomerase/histone |
| | | | AF130111 | deacetylase 3 |
| 12 | TTGGTCCTCT | 23 | AF026844 | ribosomal protein L41/ribosomal protein |
| | | | NM001007 | S4 |
| 13 | ACTTTTTCAA | 22 | | no reliable matches |
| 14 | CCTGTAATCC | 22 | | multiple matches |
| 15 | CTTCCTTGCC | 21 | X05803 | keratin 17 |
| 16 | TGTGTTGAGA | 21 | M27364/L141490 | elongation factor 1-alpha 1/elongation |
| | | | | factor 1-alpha 1-like14 |
| 17 | GCAGCCATCC | 20 | U14969/ | ribosomal protein L28/triosephosphate |
| | | | BC004230 | isomerase 1 |
| 18 | GTGGAGGGCA | 20 | U81233/U62800 | cystatin E/cystatin M |
| 19 | CACAAACGGT | 19 | L19739/ | ribosomal protein S27/sperm associated |
| | | | BC011934 | antigen 7 |
| 20 | GATGTGCACG | 19 | AA583889 | keratin 14 |
| 21 | GGATTTGGCCT | 19 | M17887 | ribosomal protein P2 |
| 22 | GGGCTGGGGT | 18 | U10248 | ribosomal protein L29 |
| 23 | TCACCCACAC | 18 | A1268626 | ribosomal protein L23 |
| 24 | CGCCGGAACA | 17 | X73974/ | ribosomal protein L4/H19, imprinted |
| | | | BC004532 | maternally exp. |
| | | | | Untransl mRNA |
| 25 | TGGTGTTGAG | 17 | X69150 | ribosomal protein S18 |
| 26 | GCCGAGGAAG | 16 | X53505 | ribosomal protein S12 |
| 27 | GTTGTGGTTA | 16 | AB021288 | beta 2-microglobulin |
| 28 | AGGTCAGGAG | 15 | | multiple matches |
| 29 | CTAAGACTTC | 15 | | no reliable matches |
| 30 | GCCTGTATGA | 15 | AA324873 | ribosomal protein S24 |
| 31 | GTGAAGGCAG | 15 | M77234/ | ribosomal protein S3a/ATP synthase, H+ |
| | | | 014710 | transporting, alpha subunit |
| 32 | TAGGTTGTCT | 15 | NM03295/AK0000 | translationally-controlled tumor protein 1/ |
| | | | 37 | hypothetical prot. FLJ20030 |
| 33 | ACCTCCACTG | 14 | AA582988 | keratinocyte differentiation associated |
| | | | | protein |
| 34 | GTGGCCACGG | 14 | AA128515 | calcium-binding protein S100 A9 |
| 35 | TAAACCTGCT | 14 | L07769 | galectin 7 |
| 36 | TGGGCAAAGC | 14 | M55409 | elongation factor-1-gamma |
| 37 | AGCACCTCCA | 13 | Z11692 | eukaryotic translation elongation factor 2 |
| 38 | GCATAATAGG | 13 | L38826 | ribosomal protein L21 |
| 39 | GCCGTGTCCG | 13 | M20020 | ribosomal protein S6 |
| 40 | TCAGATCTTT | 13 | M22146 | ribosomal protein S4 |
| 41 | GAAAACAAAG | 12 | M77663 | keratin 10 |
| 42 | TTGGCCAGGC | 12 | | multiple matches |
| 43 | AAGACAGTGG | 11 | X66699 | ribosomal protein L37a |
| 44 | CCACTGCACT | 11 | | multiple matches |
| 45 | GCGAAACCCC | 11 | | multiple matches |
| 46 | GGAGGGGGCT | 11 | X03444 | lamin A/lamin C |
| 47 | AAGGTGGAGG | 10 | L05093 | ribosomal protein L18a |
| 48 | AATAGGTCCAA | 10 | M64716 | ribosomal protein S25 |
| 49 | GAACACATCCA | 10 | S56985 | ribosomal protein L19 |
| 50 | GCAAAACCCC | 10 | | multiple matches |

^{a}Tags have been ranked by abundance, as indicated by tag count.
^{b}The accession number or numbers indicate a representative EST derived from the corresponding UniGene cluster.
No accession number has been provided for tags with either multiple matches or an EST match.
^{c}Derived from UniGene Build 108.

Analysis of SAGE Tags from Pre-Auricular Skin

Among the 4,830 tags generated from pre-auricular skin, 2,931 were found to be unique. Of these 127 unique tags (4%) appeared more than 5 times; 30% of the total amount of tags were represented by these repetitive tag sequences. Almost 50% or 2,393 of all tags appeared only once. Table 2 provides a summary of the 50 most abundant tags detected in our SAGE library constructed from pre-auricular skin. As for post-auricular skin, all but one tag (ACCTCCACTG, tag 22 in pre-auricular skin, tag 33 in post-auricular skin) could be matched to at least one gene and multiple tags could be assigned to more than one gene. Nearly all of the most abundant pre-auricular skin tags were also found to be of high copy number in post-auricular skin. Except for tag 28 (ATCCGCGAGGC, calmodulin-like skin protein) all tags in the list of the 50 most abundant pre-auricular tags were found either among the 50 most abundant tags in post-auricular or were detected in similar tag numbers. The majority of the most abundant tags in pre-auricular skin were derived from mRNAs encoded by housekeeping genes, consistent with previous SAGE studies using other tissues (Chen et al., 1998; Velculescu et al., 1997).

TABLE IV

+HZ,51
The 50 most abundant tags from a human pre-auricular SAGE
library

| | | | |
|---|---|---|---|
| Tag Sequence | Tag Count^{a} | Accession No.^{b} | UniGene match^{c} |

| | | | | |
|---|---|---|---|---|
| 1 | CCCGTCCGGA | 50 | AA010823 | ribosomal protein L13 |
| 2 | TAAACCTGCT | 40 | L07769 | galectin 7 |
| 3 | CGCCGCCGGC | 34 | U12465 | ribosomal protein L35 |
| 4 | GTGAAACCCC | 34 | | multiple matches |
| 5 | GATGTGCACG | 26 | AA583889 | keratin 14 |
| 6 | TTGGTCCTCT | 26 | AF026844/ | ribosomal protein L41/ribosomal protein |
| | | | NM001007 | S4 |
| 7 | GCCCCTGCTG | 25 | M19723 | keratin 5 |
| 8 | GCAGCCATCC | 24 | U14969/ | ribosomal protein L28/triosephosphate |
| | | | BC004230 | isomerase 1 |
| 9 | TGTGTTGAGA | 24 | M27364/L141490 | elongation factor 1-alpha 1/elongation |
| | | | | factor 1-alpha 1-like14 |
| 10 | ACGCAGGGAG | 23 | AF187554/ | glucose phosphate isomerase/histone |
| | | | AF130111 | deacetylase 3 |
| 11 | GAAAACAAAG | 23 | J04029 | keratin 10 |
| 12 | TGCACGTTTT | 23 | X03342 | ribosomal protein L32 |
| 13 | AGGCTACGGA | 21 | AA045770 | ribosomal prot. L13a |
| 14 | CGCTGGTTCC | 21 | L05092 | ribosomal protein L11 |
| 15 | GCCGAGGAAG | 21 | X53505/ | ribosomal protein S12/hypothetical protein |
| | | | AK025643 | |
| 16 | GGCAAGCCCC | 21 | AF107044/AL022 | SRY-box 21/ribosomal protein L10a |
| | | | 721 | |
| 17 | CCTGTAATCC | 20 | | multiple matches |
| 18 | GAGGGAGTTT | 20 | U14968 | ribosomal protein L27a |
| 19 | GGGCTGGGGT | 20 | U10248/ | ribosomal protein L29/sperm associated |
| | | | BC011934 | antigen 7 |
| 20 | GTGGAGGGCA | 19 | U81233/U62800 | cystatin E/cystatin M |
| 21 | TGGTGTTGAG | 19 | X69150 | ribosomal protein S18 |
| 22 | ACCTCCACTG | 18 | AA582988 | Likely ortholog of rat keratinocyte |
| | | | | differentiation associated |
| | | | | protein |
| 23 | GCCGTGTCCG | 18 | M20020 | ribosomal protein S6 |
| 24 | CGCCGGAACA | 17 | X73974/ | ribosomal protein L4/H19, impaired |
| | | | BC004532 | maternally exp. Untransl. mRNA |
| 25 | GGACCACTGA | 17 | X73460 | ribosomal protein L3 |
| 26 | TGGGCAAAGC | 17 | M55409 | elongation factor-1-gamma |
| 27 | AGGTCAGGAG | 16 | | multiple matches |
| 28 | ATCCGCGAGGC | 16 | Af172852 | calmodulin-like skin protein |
| 29 | GGATTTGGCC | 16 | M17887 | ribosomal protein P2 |
| 30 | CTAAGACTTC | 15 | | no reliable match |
| 31 | AAGGTGGAGGA | 14 | AB007175 | ribosomal protein L18a |
| 32 | GTGGCCACGG | 14 | M26311 | S100 calcium-binding protein A9 |
| 33 | AAAAAAAAAA | 13 | | multiple matches |
| 34 | CTTCCTTGCC | 13 | X05803 | keratin 17 |
| 35 | GCATAATAGG | 13 | U14967 | ribosomal protein L21 |
| 36 | TCACCCACAC | 13 | A1268626 | ribosomal protein L23 |
| 37 | TTCAATAAAA | 13 | M17886/ | ribosomal protein P1/FLJ21550 fis, clones |
| | | | AK025203 | COL06258 |
| 38 | CACAAACGGT | 12 | L19739 | ribosomal protein S27 |
| 39 | CCACTGCACT | 12 | | multiple matches |
| 40 | CCCATCCGAA | 11 | L07282 | ribosomal protein L 26 |
| 41 | GTGAAACCCT | 11 | | multiple matches |
| 42 | GTTGTGGTTA | 11 | AB021288 | beta 2-microglobulin |
| 43 | AAGGAGATGG | 10 | X15940 | ribosomal protein L31 |
| 44 | AGAAAAAAAA | 10 | AB024057/ | vascular Rab-GAP (TBC- |
| | | | X15940 | containing)/ribosomal protein L31 |
| 45 | CTGGGTTAAT | 10 | M81757 | ribosomal protein S19 |
| 46 | GACGACACGA | 10 | L05091 | ribosomal protein S28 |
| 47 | AGGCTCCTGGC | 9 | AF106911 | member 14 (BRAK) of the small in- |
| | | | | ducible cytokine subfamily B |
| 48 | ATGGCTGGTAT | 9 | X17206 | ribosomal protein S2 |
| 49 | CCAGTGGCCCG | 9 | U14971 | ribosomal protein S9 |
| 50 | CTCCTGGGCGC | 9 | M58026 | calmodulin-like 3 |

Analysis of SAGE Tags from Epidermis

5,215 SAGE tags were generated from epidermal nick biopsies, representing 2,982 unique genes. Tag distribution in this library was similar to the distribution observed in SAGE libraries from pre-and post-auricular skin libraries. The most abundant tags in the epidermal library (5 or more tags) represented 4% of the unique tags. Single tags represented 80% of the unique tags and 45% of all tags present in this SAGE library. The 50 most frequent epidermal tags are listed in Table V. Of these 50 tags, two tags did not show any gene match by comparison to the UniGene database, three tags could only be assigned to UniGene EST clusters and fourteen tags (28%) could not be attributed to a single gene. Among the seven most highly expressed genes, four different keratins (Keratin 1, 10, 5 and 14) were found, typically expressed in epidermal keratinocytes. The genes for the intermediate filament proteins Keratin 5 and 14 are known to be highly expressed in the basal layer of the epidermis, whereas Keratin 1 and 10 are predominantly found in the differentiating keratinocytes of the suprabasal layers of the epidermis. Tags from mRNA encoding filaggrin, which cross-links keratin, and also plakoglobin, a cross-linker of intermediate filaments and the dense plaques of desmosomes, are also among the 50 most abundant tags. The remainder of these tags in this table are largely derived from genes known to be expressed in many different tissues.

TABLE V

The 50 most abundant tags
from a human epidermal SAGE library

| | | | |
|---|---|---|---|
| | Tag | | |
| Tag Sequence | Count^{a} | Accession No.^{b} | UniGene match^{c} |

| | | | | |
|---|---|---|---|---|
| 1 | GAAAACAAAG | 77 | M77663 | keratin 10 |
| 2 | CCCGTCCGGA | 66 | AA010823 | ribosomal protein L13 |
| 3 | GCCCGTGCTG | 52 | M21389 | keratin 5 |
| 4 | GATGTGCACG | 48 | AA583889 | keratin 14 |
| 5 | ACTTTTTCAA | 42 | | no reliable matches |
| 6 | ACAGCGGCAA | 40 | M77830 | desmoplakin I |
| 7 | ACATTTCAAAG | 39 | AA024512 | keratin 1 |
| 8 | CGCCGCCGGC | 39 | U12465 | ribosomal protein L35 |
| 9 | TAAACCTGCT | 39 | L07739 | galectin 7 |
| 10 | GGATTTGGCCT | 33 | M17887 | ribosomal protein P2 |
| 11 | GTGAAACCCC | 33 | | multiple matches |
| 12 | GTTGTGGTTAA | 32 | AB021288 | beta 2-microglobulin |
| 13 | GGGCTGGGGTC | 30 | U10248/AF04743 | ribosomal protein L29/sperm associated |
| | | | 7 | antigen 7 |
| 14 | ACCTCCACTGG | 25 | AA582988 | Keratinocyte differentiation associated |
| | | | | protein |
| 15 | CCACAGGAGAA | 25 | AJ251830 | p53-induced protein PIGPC1 |
| 16 | ATCCGCGAGGC | 24 | Af172852 | calmodulin-like skin protein |
| 17 | CCTGTAATCC | 23 | | multiple matches |
| 18 | GCAGCCATCCG | 21 | U14969/ | ribosomal protein L28/triosephosphate |
| | | | BC004230 | isomerase 1 |
| 19 | GGACCACTGAA | 20 | M90054 | ribosomal protein L3 |
| 20 | TGTGTTGAGA | 20 | M27364/L141490 | elongation factor 1-alpha 1/elongation |
| | | | | factor 1-alpha 1-like 14 |
| 21 | AGAAAAAAAAA | 19 | | multiple matches |
| 22 | CGCTGGTTCC | 19 | L05092 | ribosomal protein L11 |
| 23 | GAGGGAGTTTC | 19 | U14968 | ribosomal protein L27a |
| 24 | GGCCGCGTTCG | 19 | M13932 | ribosomal protein S17 |
| 25 | GCCGAGGAAG | 18 | X53505/ | ribosomal protein S12/hypothetical |
| | | | AK025643 | protein |
| 26 | GTGTGGGGGG | 18 | Z68228 | plakoglobin |
| | C | | | |
| 27 | AAGGTGGAGGA | 17 | L05093 | ribosomal protein L18a |
| 28 | GAGAGCTAACT | 17 | M60502 | filaggrin |
| 29 | GCCGTGTCCG | 17 | M20020 | ribosomal protein S6 |
| 30 | GGCAAGCCCCA | 17 | AF107044/AL022 | SRY-box 21/ribosomal protein L10a |
| | | | 721 | |
| 31 | ATGGCTGGTAT | 16 | AL031671 | ribosomal protein S2 |
| 32 | GCCTTCTGGAT | 16 | AA733153 | protein phosphatase 2 |
| 33 | AAAAAAAAAA | 15 | | multiple matches |
| 34 | CAGGTTTCATA | 15 | AF106911 | member 14 (BRAK) of the small |
| | | | | inducible cytokine subfamily B |
| 35 | CCACTGCACT | 13 | | multiple matches |
| 36 | CCAGAACAGAC | 15 | L05095/L16991 | ribosomal protein L30/deoxythymidylate |
| | | | | kinase |
| 37 | TTCAATAAAAA | 15 | | multiple matches |
| 38 | ATTGAGAAGC | 14 | | no reliable matches |
| 39 | TTGGTCCTCTG | 14 | AF026844/ | ribosomal protein L41/tribosomoal |
| | | | NM001007 | protein S4 |
| 40 | AGGCTCCTGGC | 13 | AF106911 | member 14 (BRAK) of the small |
| | | | | inducible cytokine subfamily B |
| 41 | TAGGTTGTCTA | 13 | NM03295/AK000 | translationally-controlled tumor protein |
| | | | 037 | 1/hypothetical prot. FLJ20030 |
| 42 | GGAGGCTGAGG | 12 | | multiple matches |
| 43 | AATCTTGTTT | 11 | BC004493 | hypothetical gene ZD52F10 |
| 44 | ACCTGGAGGGG | 11 | | ESTs |
| 45 | ATAATTCTTT | 11 | AK021540/AA147 | cDNA FLJ11778 fis, clone |
| | | | 325 | HEMBA1005911/ribosomal protein S29 |
| 46 | CTGGGTTAAT | 11 | M81757 | ribosomal protein S19 |
| 47 | CCAGTGGCCC | 10 | AI064904 | ribosomal protein S9 |
| 48 | GCGAAACCCC | 10 | | multiple matches |
| 49 | TCAGATCTTT | 10 | M22146 | ribosomal protein S4 |
| 50 | ACGCAGGGAG | 9 | AF187554/ | glucose phosphate isomerase/histone |
| | | | AF130111 | deacetylase 3 |

### EXAMPLE 3

In this SAGE analysis of mRNA profiles in human skin, over 15,000 tags were identified, representing mRNAs from more than 6,500 different genes. These mRNAs in full thickness skin were identified in poly(A⁺)RNA isolated from chronically sun-exposed pre-auricular skin and sun-protected post-auricular skin, obtained from a patient with sundamage undergoing elective facial plasty. By choosing this model of pre-and post-auricular skin, at least some of the limitations of other model systems were circumvented, including the inherent difficulties of mice to study solar elastosis. Using sun-exposed human skin also allows to study the effects of natural sunlight, rather then having to differentiate between the effects of the different components of sunlight. A recent study by Brown et al. (Brown et al., 2000) reported, for example, that common fluorescent sunlamps are inadequate substitutes for natural sunlight. Moreover, cell culture models rarely give a satisfactory representation of the different cell types as well as the three-dimensional structure of tissues with the consequence of, for example, neglecting the interactions between cell types. Additionally, the skin biopsies in the present study were taken from adjacent sites of the face in an attempt to minimize phenotypic differences in skin from different regions of the body. However, using pre- and post-auricular human skin, the possibilities of controlling and influencing experimental conditions such as total duration of sun exposure, are limited. Furthermore, we are aware that our model reflects changes in mRNA steady state levels that are due to many years of sun exposure rather than representing alterations caused by a controlled and limited exposure to UV light.

Full thickness human skin contains a variety of different cell populations, the most abundant cell type in human skin being epidermal derived keratinocytes. It would be expected, therefore, that an mRNA profile obtained from full thickness skin would reflect a spectrum of mRNAs derived largely from keratinocytes, and this is indeed the result obtained. In comparing the fifty most abundant mRNAs in both pre-and post-auricular skin to the mRNA profile identified in a SAGE library from epidermal nick biopsies, it is clear that many of these tags are derived from mRNAs encoding proteins typically found in epidermis. These include keratins, galectin 7 and calmodulin-like skin protein.

A similar analysis of tags obtained from a human skin fibroblast SAGE library (data not shown) revealed several mRNAs expected to be among the most abundant in skin fibroblasts. These include the mRNAs encoding proα1(1), proα2(I) and proα1(III) collagen and several matrix metalloproteinases. Tags derived from these mRNAs were not observed in our full thickness skin libraries, supporting the conclusion that most of the abundant mRNAs observed in this SAGE analysis of full thickness skin are derived from epidermal keratinocytes.

Tables I and II list tags derived from 34 different genes that are either increased or decreased in abundance between pre-and post-auricular skin. These changes in tag numbers are a direct reflection of changes in steady state levels for mRNAs from which these tags were derived, which indirectly reflects changes in gene expression. The underlying assumption is that altered mRNA levels will be reflected in changes in the amount of proteins these mRNAs encode.

Of the 34 different mRNAs that are represented by at least a 4-fold difference in abundance between pre- and post-auricular skin, 6 of these mRNAs encode ribosomal proteins and 2 mRNAs encode translation initiation factor proteins. We have assumed in this study that these changes in ribosomal and initiation factor protein mRNA reflect overall changes in protein synthesis associated with chronic sun-exposure. Tags derived from mRNAs encoding ribosomal proteins are commonly present in most SAGE studies and most authors attach no particular functional significance to the appearance of these tags.

Four tags in Tables I and II were shown to have multiple matches. Three of these tags correspond to stretches of poly(A). As SAGE tags are constructed from the 3′-end of mRNAs, these poly(A) sequences almost certainly represent either stretches of poly(A) within the 3′-untranslated region (UTR) of one or more mRNAs; or poly(A) tails added post-transcriptionally to the 3′-end of the 3′UTR of mRNAs. In either example, the tag match to these common sequences in most mRNA precludes a more definite identification of the mRNA from which these tags were derived. Similarly, a UniGene match for a tag in Table II identified as hypothetical protein (AF151075) represents a previously identified EST cluster of no known function. An EST with limited homology to an mRNA predicted to synthesize protein AAB542440 is also an EST encoding a protein of unknown function. The two tags in Table I that were not identified in GenBank represent mRNAs not previously identified as ESTs.

Of the remaining tags encoding 18 different mRNAs, it is striking that the proteins these mRNAs encode represent a functionally diverse group of proteins, largely confined to the epidermis. Taken together, these changes suggest a defense mechanism of skin and specifically of the epidermis against chronic exposure to UV irradiation that includes a sustained inflammatory reaction, as indicated by the elevated levels of MIF. Furthermore IGFBP-6, CLSP and EFABP, all of which have been implicated in keratinocyte differentiation, showed increased mRNA steady state levels and additionally the increased level of apoptosis inhibition by the Bcl-2 antagonist and Bax-inhibitor 1 implies an altered keratinocyte proliferation-differentiation cycle in sun-damaged skin. Moreover, as Ca²⁺ levels are known to be an important factor for this cycle switch in keratinocytes, it is not surprising that the mRNA levels encoding several Ca²⁺ binding proteins are also altered in sun-damaged skin.

The pre-and post-auricular SAGE libraries described herein were constructed from skin samples obtained from a 55 year old female donor at the time she was undergoing elective facial plasty. This pre-auricular skin sample therefore represented skin subject to many decades of repeated sun exposure. Few studies have addressed the biosynthetic consequences of chronic and repeated sun exposure. Voorhees and his colleagues, for example, have proposed an attractive hypothesis of UV-induced, MAP-kinase mediated activation of matrix metalloproteinases as the underlying mechanism for the aberrant remodeling of collagens and other components of dermal connective tissue during repeated sun exposure.

In summary, the SAGE analysis performed in connection with the present invention is the first attempt to obtain a comprehensive profile of biosynthetic changes in full thickness human skin associated with chronic sun exposure. Eighteen different mRNAs were identified from a total of unique 6,500 transcripts analyzed that have significantly altered steady state levels associated with chronic sun exposure.

While the present invention has been described herein with some specificity, and with reference to certain preferred embodiments thereof, those of ordinary skill in the art will recognize numerous variations, modifications and substitutions of that which has been described which can be made, and which are within the scope and spirit of the invention. It is intended that all of these modifications and variations be within the scope of the present invention as described and claimed herein, and that the inventions be limited only by the scope of the claims which follow, and that such claims be interpreted as broadly as is reasonable. Throughout this application, various publications have been cited. The entireties of each of these publications are hereby incorporated by reference herein:

What is claimed is:

1. A personal care method of detecting photodamage comprising the steps of:
(A) using at least one marker of photodamage, the marker selected from one or more sequences selected from the group consisting of sequence No. 51, sequence No. 52, sequence No. 53, sequence No. 54, sequence No. 55, sequence No. 56, sequence No. 57, sequence No. 58, sequence No. 59, sequence No.60, sequence No. 61, sequence No. 62, sequence No. 63, sequence No. 64, sequence No. 65, sequence No. 66, sequence No. 67, sequence No. 68, and sequence No. 69;
and
(B) detecting a change in the marker to determine the presence of photodamage.

2. The method of claim 1 wherein the detecting step (b) comprises the further step of:
(b1) comparing a first skin sample with a second skin sample to determine whether there is a change in the marker.

3. A personal care method for detecting a skin condition in an epidermal or dermal or total skin sample, the method comprising the steps of:
a) determining a first gene expression of a predetermined skin sample having a known skin condition;
b) determining a second gene expression of a second predetermined skin sample having no known skin condition; and
c) identifying a plurality of markers of a change in the first and second gene expressions, so that the plurality of markers identify the known skin condition.

4. The method of claim 3 wherein the identifying step (c) further comprises the step of:
(c1) determining the marker by assessing a change in gene expression between the first gene expression and the second gene expression.

5. The method of claim 3 wherein the known skin condition is selected from the conditions comprising: photodamage, aging, dry skin, and oily skin.

6. The method of claim 3 wherein the known skin condition is photodamage and the plurality of markers are selected from the group consisting of sequence No. 51, sequence No. 52, sequence No. 53, sequence No. 54, sequence No. 55, sequence No. 56, sequence No. 57, sequence No. 58, sequence No. 59, sequence No. 60, sequence No. 61, sequence No. 62, sequence No. 63, sequence No.64, sequence No.65, sequence No.66, sequence No.67, sequence No.68, and sequence No.69.


SEQUENCE LISTING (34 sequences)

SEQ ID NO: 1 - DNA, 11 bp, Artificial Sequence
  Description of Artificial Sequence Seq.#51 of Table I
caaaaaaaaaa

SEQ ID NO: 2 - DNA, 10 bp, Artificial Sequence
  Description of Artificial Sequence Seq.# 52 of Table I
acgttaaaga

SEQ ID NO: 3 - DNA, 11 bp, Artificial Sequence
  Description of Artificial Sequence Seq.# 53 of Table I
acatcatcgat

SEQ ID NO: 4 - DNA, 11 bp, Artificial Sequence
  Description of Artificial Sequence Seq.# 54 of Table I
actccaaaaaa

SEQ ID NO: 5 - DNA, 11 bp, Artificial Sequence
  Description of Artificial Sequence Seq.# 55 of Table I
gaaatacagtt

SEQ ID NO: 6 - DNA, 11 bp, Artificial Sequence
  Description of Artificial Sequence Seq.# 56 of Table I
gccaggagcta

SEQ ID NO: 7 - DNA, 11 bp, Artificial Sequence
  Description of Artificial Sequence Seq.# 57 of Table I
ctcctcacctg

SEQ ID NO: 8 - DNA, 11 bp, Artificial Sequence
  Description of Artificial Sequence Seq.# 58 of Table I
caataaactga

SEQ ID NO: 9 - DNA, 11 bp, Artificial Sequence
  Description of Artificial Sequence Seq.# 59 of Table I
cagctcactga

SEQ ID NO: 10 - DNA, 11 bp, Artificial Sequence
  Description of Artificial Sequence Seq.# 60 of Table I
caggacctggt

SEQ ID NO: 11 - DNA, 11 bp, Artificial Sequence
  Description of Artificial Sequence Seq.# 61 of Table I
cccaacgcgct

SEQ ID NO: 12 - DNA, 11 bp, Artificial Sequence
  Description of Artificial Sequence Seq.# 62 of Table I
ccctggcaatg

SEQ ID NO: 13 - DNA, 11 bp, Artificial Sequence
  Description of Artificial Sequence Seq.# 63 of Table I
ctgccaagttg

SEQ ID NO: 14 - DNA, 11 bp, Artificial Sequence
  Description of Artificial Sequence Seq.# 64 of Table I
gcaaaaccccg

SEQ ID NO: 15 - DNA, 11 bp, Artificial Sequence
  Description of Artificial Sequence Seq.# 65 of Table I
ggaaaaaaaaa

SEQ ID NO: 16 - DNA, 11 bp, Artificial Sequence
  Description of Artificial Sequence Seq.# 66 of Table I
ggggcagggcc

SEQ ID NO: 17 - DNA, 11 bp, Artificial Sequence
  Description of Artificial Sequence Seq.# 67 of Table I
gtgcactgagc

SEQ ID NO: 18 - DNA, 11 bp, Artificial Sequence
  Description of Artificial Sequence Seq.# 68 of Table I
tctcccacacc

SEQ ID NO: 19 - DNA, 11 bp, Artificial Sequence
  Description of Artificial Sequence Seq.# 69 of Table I
cggggtggccg

SEQ ID NO: 20 - DNA, 11 bp, Artificial Sequence
  Description of Artificial Sequence Seq.# 70 of Table II
acatttcaaag

SEQ ID NO: 21 - DNA, 11 bp, Artificial Sequence
  Description of Artificial Sequence Seq.# 71 of Table II
cagctatttca

SEQ ID NO: 22 - DNA, 11 bp, Artificial Sequence
  Description of Artificial Sequence Seq.# 72 of Table II
ggcccctcacc

SEQ ID NO: 23 - DNA, 11 bp, Artificial Sequence
  Description of Artificial Sequence Seq.# 73 of Table II
atccgcgaggc

SEQ ID NO: 24 - DNA, 11 bp, Artificial Sequence
  Description of Artificial Sequence Seq.# 74 of Table II
aacgcggccaa

SEQ ID NO: 25 - DNA, 11 bp, Artificial Sequence
  Description of Artificial Sequence Seq.# 75 of Table II
gagcagcgccc

SEQ ID NO: 26 - DNA, 11 bp, Artificial Sequence
  Description of Artificial Sequence Seq.# 76 of Table II
aagaagataga

SEQ ID NO: 27 - DNA, 11 bp, Artificial Sequence
  Description of Artificial Sequence Seq.# 77 of Table II
taaaaaaaaaa

SEQ ID NO: 28 - DNA, 11 bp, Artificial Sequence
  Description of Artificial Sequence Seq.# 78 of Table II
tcagacttttg

SEQ ID NO: 29 - DNA, 11 bp, Artificial Sequence
  Description of Artificial Sequence Seq.# 79 of Table II
ttggtgaagga

SEQ ID NO: 30 - DNA, 11 bp, Artificial Sequence
  Description of Artificial Sequence Seq.# 80 of Table II
aactaacaaaa

SEQ ID NO: 31 - DNA, 11 bp, Artificial Sequence
  Description of Artificial Sequence Seq.# 81 of Table II
caataaatgtt

SEQ ID NO: 32 - DNA, 11 bp, Artificial Sequence
  Description of Artificial Sequence Seq.# 82 of Table II
gctcccagact

SEQ ID NO: 33 - DNA, 11 bp, Artificial Sequence
  Description of Artificial Sequence Seq.# 83 of Table II
ggaagtttcga

SEQ ID NO: 34 - DNA, 11 bp, Artificial Sequence
  Description of Artificial Sequence Seq.# 84 of Table II
tcaaaaatata